(19) United States
(12) Patent Application Publication
(10) Pub. No.: US 2026/0345340 A1
(43) Pub. Date: Nov. 5, 2026

(54) EXPANDABLE SPINAL IMPLANT

(71) Applicant: MEDTRONIC HOLDING COMPANY SARL, Tolochenaz (CH)

(72) Inventor: Gil Machado, Galway (IE)

(21) Appl. No.: 19/475,869

(22) PCT Filed: Mar. 28, 2024
(86) PCT No.: PCT/EP2024/058553

§ 371 Date: Oct. 16, 2025

Related U.S. Application Data
(60) Provisional application No. 63/460,208, filed on Apr. 18, 2023.

(51) Int. Cl. A61F 2/44; A61F 2/30

(52) U.S. Cl. CPC A61F 2/447; A61F 2002/30433; A61F 2002/30471; A61F 2002/30579

(57) ABSTRACT

An expandable spinal implant is provided. One or more of the expandable spinal implants can be configured for insertion into an interior of a vertebral body of a vertebrae to facilitate reinforcement of the vertebral body, and/or also for insertion into a disc space between adjacent vertebral bodies for use as an interbody spinal implant facilitating fusion between the adjacent vertebral bodies. The expandable spinal implant can include a first upper expansion portion that is outwardly expandable laterally and upwardly, and a second lower expansion portion that is outwardly expandable laterally and downwardly in similar fashion.

### FIELD

The present technology is generally related to medical devices, and more specifically to an expandable spinal implant.

### BACKGROUND

Spinal disorders of a patient oftentimes result from a weakened vertebral body or bodies. To illustrate, a vertebral body can be weakened by compression factures or other types of deterioration. For example, the cancellous bone of the vertebral body can deteriorate to an extent that causes subsidence of cortical bone surrounding the cancellous bone. Such deterioration and corresponding subsidence can cause deleterious spinal disorders. Therefore, there is a need for a spinal implant that can compensate for deterioration of the cancellous bone of a vertebral body.

### SUMMARY

The techniques of this disclosure generally relate to an expandable spinal implant. As discussed below, the expandable spinal implant can be inserted into a vertebral body, and adjusted from an unexpanded configuration to an expanded configuration within the vertebral body. When in the expanded configuration, the expandable spinal implant can serve as a buttress for reinforcing the cortical bone of the vertebral body.

In one aspect, the present disclosure provides an expandable spinal implant including a distal first end, an opposite proximal second end, a major mid-longitudinal axis extending through the distal first end and the proximal second end, and a minor mid-longitudinal axis extending perpendicularly to the mid-longitudinal axis, a first reference plane extending through and along the major mid-longitudinal axis; a distal end portion adjacent the distal first end, and a proximal end portion adjacent the proximal first end, the distal end position and the proximal portion being moveable toward one another; a first leg portion, a second leg portion, a third leg portion, and a fourth leg portion, each of the first leg portion, the second leg portion, the third leg portion, and the fourth leg portion including a first end and an opposite second end, the first end of the first leg portion being attached relative to the distal end portion on a first side of the first plane, the first end of the second leg portion being attached relative to the distal end portion on a second side of the first plane, the first end of the third leg portion being attached relative to the proximal end portion on the second side of the first plane, and the first end of the fourth leg portion being attached relative to the proximal end portion on the first side of the first plane, a first joint portion positioned between the first leg portion and the fourth leg portion, the second ends of the first leg portion and the fourth leg portion being attached on opposite sides of the first joint portion, a second joint portion positioned between the second leg portion and the third leg portion, the second ends of the second leg portion and the third leg portion being attached on opposite sides of the second joint portion, a first arm portion, a second arm portion, a third arm portion, and a fourth arm portion, each of the first arm portion, the second arm portion, the third arm portion, and the fourth arm portion including a first end and an opposite second end, the first ends of the first arm portion and the second arm portion being attached relative to one another, the first ends of the third arm portion and the fourth arm portion being attached relative to one another, the second ends of the first arm portion and the fourth arm portion being attached relative to the first joint portion, and the second ends of the second arm portion and the third arm portion being attached relative to the second joint portion, where, when the distal end portion and the proximal end portion are moved toward one another, the first joint portion is forced outwardly in a first lateral direction and upwardly relative to the major mid-longitudinal axis, the second joint portion is forced outwardly in a second lateral direction and upwardly relative to the major mid-longitudinal axis; and where outward and upward movement of the first joint portion moves the second ends of the first arm portion and the fourth arm portion outwardly in the first lateral direction and upwardly, and outward and upward movement of the second joint portion moves the second ends of the second arm portion and the third arm portion outwardly in the second lateral direction and upwardly, the outward and upward movement of the second end of the first arm portion and the outward and upward movement of the second end of the second arm portion moving the second ends of the first arm portion and the second arm portion apart from one another, and the outward and upward movement of the second end of the third arm portion and the outward and upward movement of the second end of the fourth arm portion moving the second ends of the third arm portion and the fourth arm portion apart from one another.

In another aspect, the present disclosure provides an expandable spinal implant configured to be move from an unexpanded first configuration to an expanded second configuration, the expandable spinal implant including a distal first end, an opposite proximal second end, a major mid-longitudinal axis extending through the distal first end and the proximal second end, and a minor mid-longitudinal axis extending perpendicularly to the mid-longitudinal axis; a distal end portion adjacent the distal first end, and a proximal end portion adjacent the proximal first end, the distal end position and the proximal portion being moveable toward one another; a first leg portion, a second leg portion, a third leg portion, and a fourth leg portion each including a first end and an opposite second end, the first end of the first leg portion being attached relative to a first portion of the distal end portion, the first end of the second leg portion being attached relative to a second portion of the distal end portion, the first end of the third leg portion being attached relative to a first portion of the proximal end portion, and the first end of the fourth leg portion being attached relative to a second portion of the proximal end portion, a first joint portion positioned between the first leg portion and the fourth leg portion, the second ends of the first leg portion and the fourth leg portion being attached on opposite sides of the first joint portion, a second joint portion positioned between the second leg portion and the third leg portion, the second ends of the second leg portion and the third leg portion being attached on opposite sides of the second joint portion, a first arm portion, a second arm portion, a third arm portion, and a fourth arm portion each including a first end and an opposite second end, the first ends of the first arm portion and the second arm portion being pivotally attached relative to one another, the first ends of the third arm portion and the fourth arm portion being pivotally attached relative to one another, the second ends of the first arm portion and the fourth arm portion being attached relative to the first joint portion, and the second ends of the second arm portion and the third arm portion being attached relative to the second joint portion, where, when the distal end portion and the proximal end portion are moved toward one another, the first joint portion is forced outwardly in a first lateral direction and upwardly relative to the major mid-longitudinal axis due to compression of the first leg portion and the fourth leg portion between the distal end portion and the proximal end portion, and the second joint portion is forced outwardly in a second lateral direction and upwardly relative to the major mid-longitudinal axis due to compression of the second leg portion and the third leg portion between the distal end portion and the proximal end portion; and where outward and upward movement of the first joint portion moves the second ends of the first arm portion and the fourth arm portion outwardly in the first lateral direction and upwardly, and outward and upward movement of the second joint portion moves the second ends of the second arm portion and the third arm portion outwardly in the second lateral direction and upwardly, movement of the first ends of the first arm portion and the fourth arm portion, and movement of the first ends of the second arm portion and the third arm portion moves the expandable spinal implant from the unexpanded first configuration to the expanded second configuration.

In yet another aspect, the present disclosure provides an expandable spinal implant configured to be move from an unexpanded first configuration to an expanded second configuration, the expandable spinal implant including a distal first end, an opposite proximal second end, a major mid-longitudinal axis extending through the distal first end and the proximal second end, and a minor mid-longitudinal axis extending perpendicularly to the mid-longitudinal axis; a distal end portion adjacent the distal first end, and a proximal end portion adjacent the proximal first end, the distal end position and the proximal portion being moveable toward one another; a first leg portion, a second leg portion, a third leg portion, and a fourth leg portion each including a first end and an opposite second end, the first end of the first leg portion being attached relative to the distal end portion, the first end of the second leg portion being attached relative to the distal end portion, the first end of the third leg portion being attached relative to the proximal end portion, and the first end of the fourth leg portion being attached relative to the proximal end portion, a first joint portion including a first side portion and a second side portion, the second ends of the first leg portion and the fourth leg portion being respectively attached relative to the first side portion and the second side portion of the first joint portion, a second joint portion including a first side portion and a second side portion, the second ends of the second leg portion and the third leg portion being respectively attached relative to the first side portion and the second side portion of the second joint portion, a first arm portion, a second arm portion, a third arm portion, and a fourth arm portion each including a first end and an opposite second end, the first ends of the first arm portion and the second arm portion being attached relative to one another, the first ends of the third arm portion and the fourth arm portion being attached relative to one another, the second ends of the first arm portion and the fourth arm portion being attached relative to the first joint portion, and the second ends of the second arm portion and the third arm portion being attached relative to the second joint portion, where, when the distal end portion and the proximal end portion are moved toward one another, the first joint portion is forced outwardly in a first lateral direction and upwardly relative to the major mid-longitudinal axis, and the second joint portion is forced outwardly in a second lateral direction and upwardly relative to the major mid-longitudinal axis; and where outward and upward movement of the first joint portion moves the second ends of the first arm portion and the fourth arm portion outwardly in the first lateral direction and upwardly, and outward and upward movement of the second joint portion moves the second ends of the second arm portion and the third arm portion outwardly in the second lateral direction and upwardly, movement of the first ends of the first arm portion and the fourth arm portion, and movement of the first ends of the second arm portion and the third arm portion moves the expandable spinal implant from the unexpanded first configuration to the expanded second configuration.

An expandable spinal implant is provided. One or more of the expandable spinal implants can be configured for insertion into an interior of a vertebral body of a vertebrae to facilitate reinforcement of the vertebral body, and/or also for insertion into a disc space between adjacent vertebral bodies for use as an interbody spinal implant facilitating fusion between the adjacent vertebral bodies. The expandable spinal implant can include a first upper expansion portion that is outwardly expandable laterally and upwardly, and a second lower expansion portion that is outwardly expandable laterally and downwardly in similar fashion. The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The techniques of this disclosure generally relate to an expandable spinal implant.

FIG. 1A is a top plan view of an embodiment of an expandable spinal implant of the present disclosure with the expandable spinal implant in an unexpanded configuration;

FIG. 1B is a top plan view similar to FIG. 1A and detailing additional features of the expandable spinal implant FIG. 1A in the unexpanded configuration;

FIG. 1C is a top, partially phantom, plan view similar to FIG. 1A and detailing additional features of the expandable spinal implant of FIG. 1A in the unexpanded configuration;

FIG. 1D is a top, partially phantom, plan view similar to FIG. 1A and detailing additional features of the expandable spinal implant of FIG. 1A in the unexpanded configuration;

FIG. 1E is a top, partially phantom, plan view similar to FIG. 1A and detailing additional features of the expandable spinal implant of FIG. 1A in the unexpanded configuration;

FIG. 2 is bottom plan view of the expandable spinal implant of FIG. 1A in the unexpanded configuration;

FIG. 3 is first side cross-sectional view of the expandable spinal implant of FIG. 1A in the unexpanded configuration taken along Line 3-3 of FIG. 1A;

FIG. 4 is a second side cross-sectional view of the expandable spinal implant of FIG. 1A in the unexpanded configuration taken along long Line 4-4 of FIG. 2;

FIG. 5 is an end cross-sectional view of the expandable spinal implant of FIG. 1A in the unexpanded configuration taken along Line 5-5 of FIG. 3;

FIG. 6 is an end cross-sectional view of the expandable spinal implant of FIG. 1A in the unexpanded configuration taken along Line 6-6 of FIG. 4;

FIG. 7 is a first cross-sectional view of the expander of the expandable spinal implant of FIG. 1A;

FIG. 8 is a second cross-sectional view of the expander of the expandable spinal implant of FIG. 1A;

FIG. 9 is a top plan view of the expandable spinal implant of FIG. 1A in an expanded configuration;

FIG. 10 is a side cross-sectional view of the expandable spinal implant of FIG. 1A in the expanded configuration taken along Line 10-10 of FIG. 9; and

FIG. 11 is an end cross-sectional view of the expandable spinal implant of FIG. 1A in the expanded configuration taken along Line 11-11 of FIG. 10.

### DETAILED DESCRIPTION

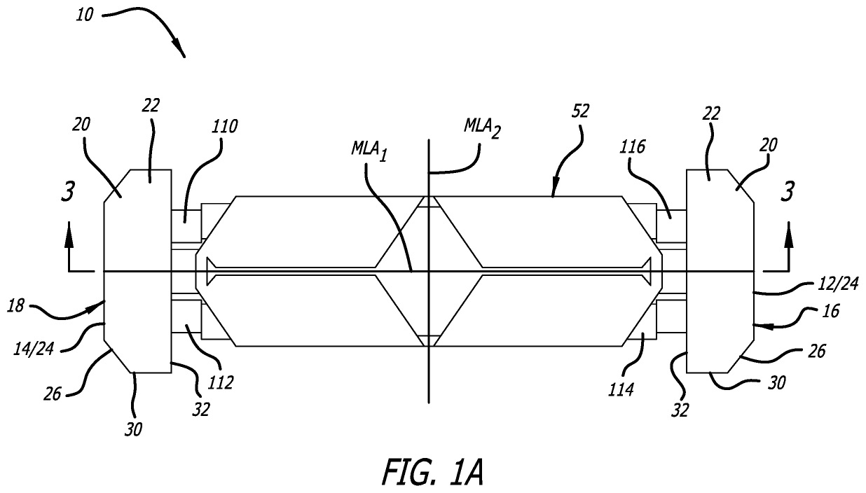

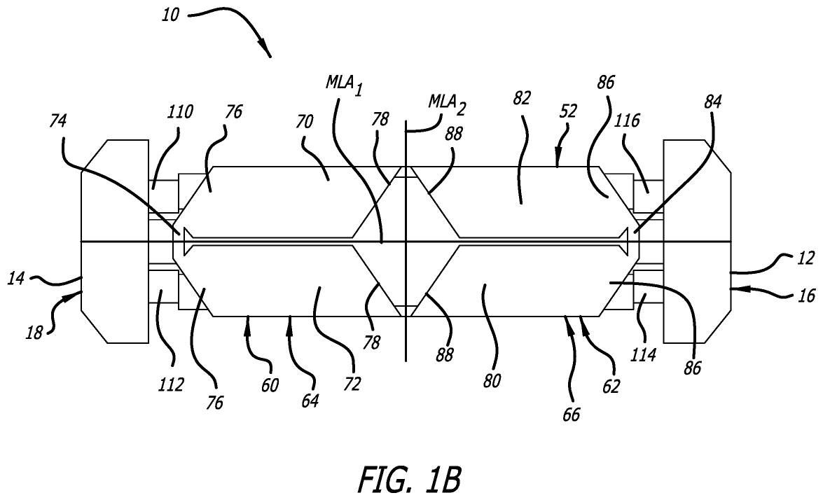

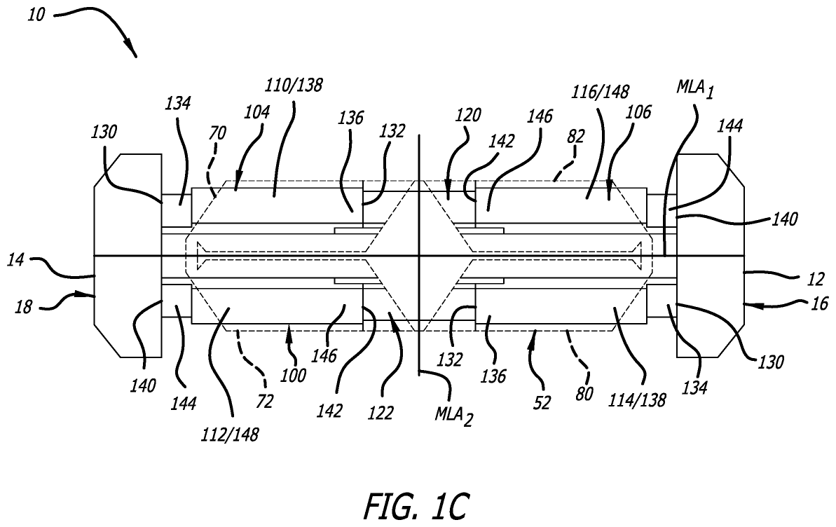

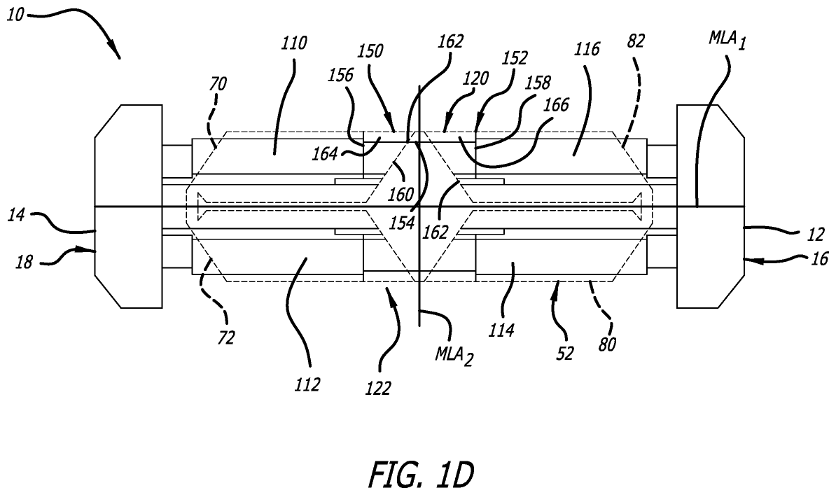

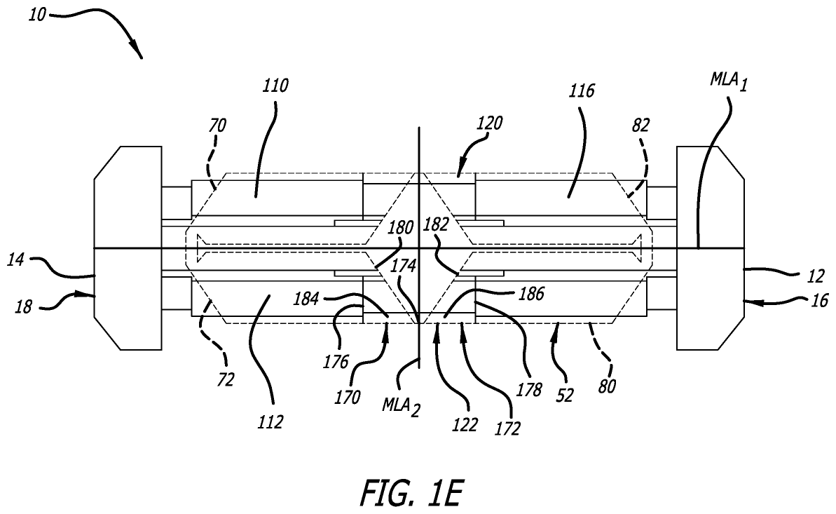

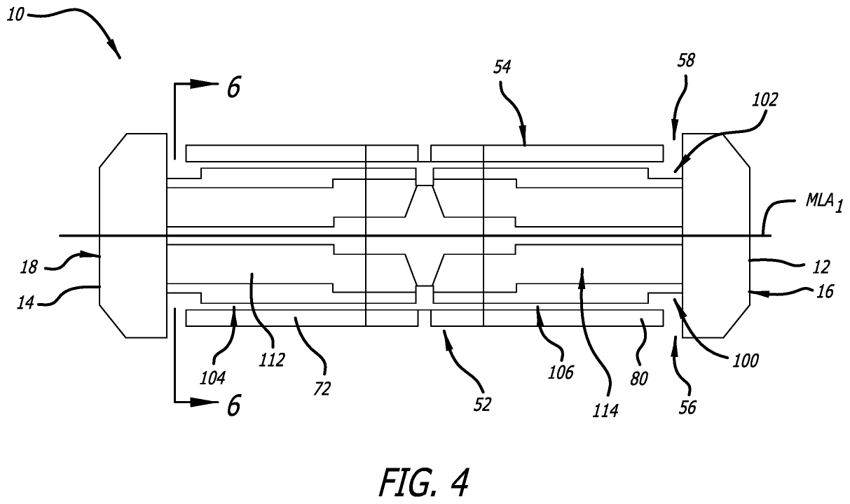

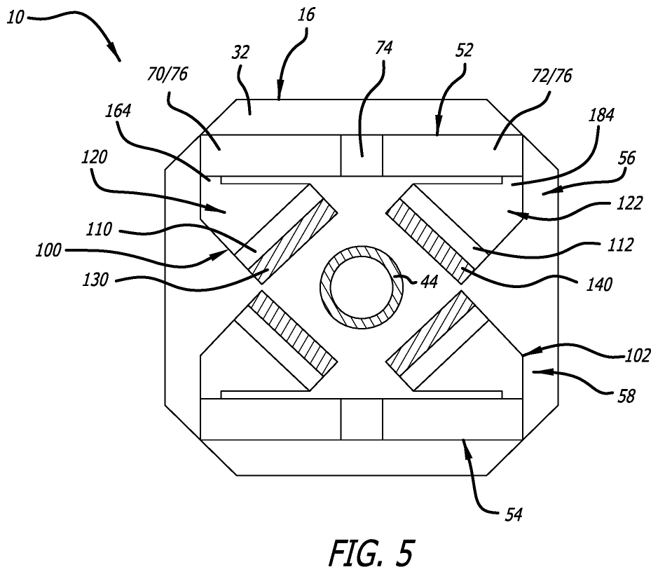

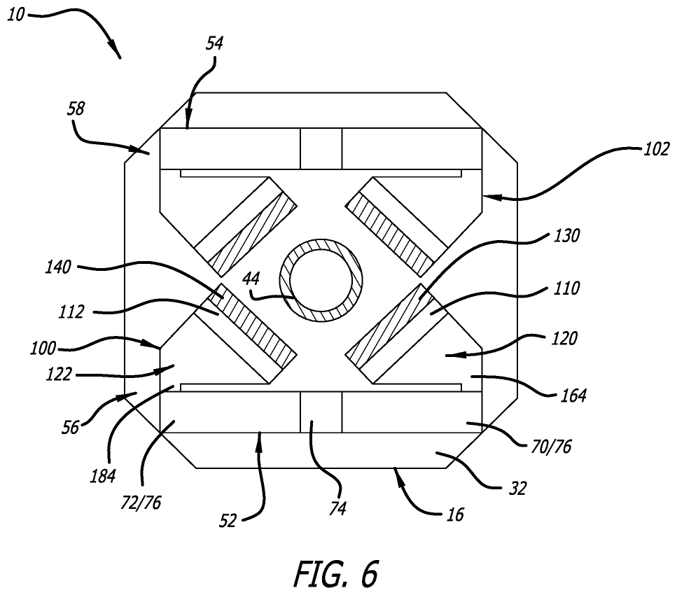

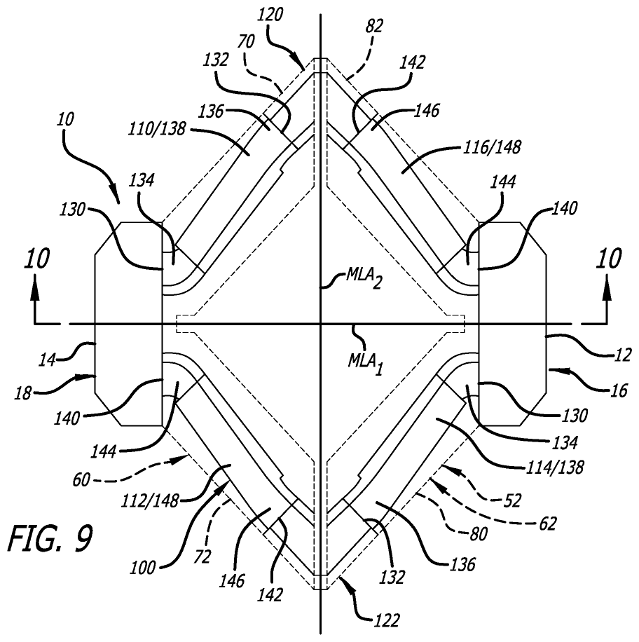

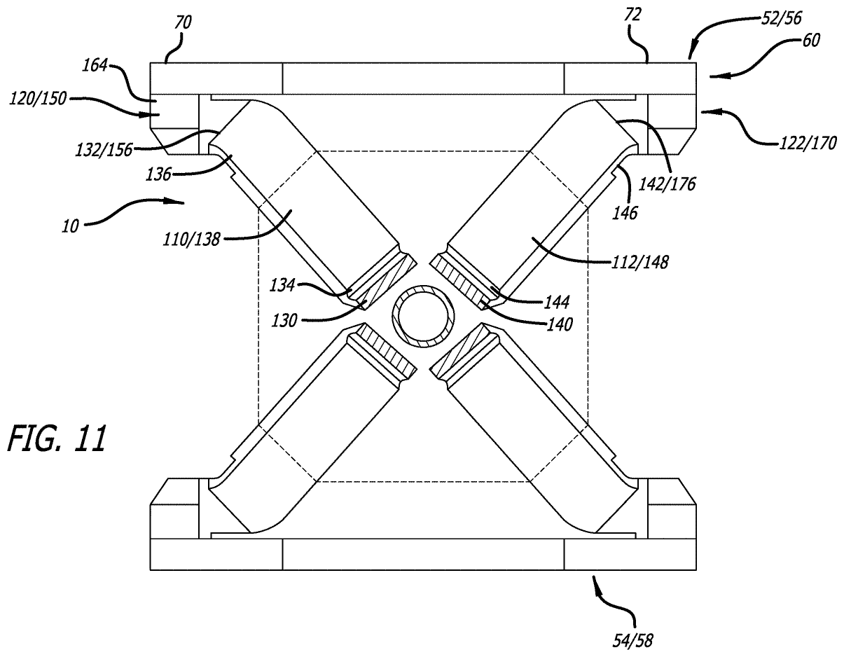

An embodiment of an expandable spinal implant of the present disclosure is generally indicated by the numeral 10 in FIGS. 1A-6 and 9-11. The spinal implant 10 is expandable between an unexpanded first configuration (FIGS. 1A-6) and an expanded second configuration (FIGS. 9-11). As discussed below, portions of the spinal implant 10 are expandable outwardly in lateral and upward/downward directions.

The spinal implant 10 can be configured for insertion into an interior of a vertebral body of a vertebrae via a cannula (not shown) to facilitate reinforcement of the vertebral body, and/or also for insertion into a disc space between adjacent vertebral bodies via the cannula for use as an interbody spinal implant facilitating fusion between the adjacent vertebral bodies. To illustrate, when used to facilitate reinforcement of the vertebral body, the cannula can be inserted via transpedicular or extrapedicular approaches relative to the vertebrae and into the vertebral body. A drill (not shown) can be used to facilitate placement of the cannula through the pedicle and into the interior of the vertebral body. After placement thereof, the cannula provides access to the interior of the vertebral body. To facilitate insertion, the spinal implant 10 can be inserted through the cannula and into the interior of the vertebral body or into the disc space between the adjacent vertebral bodies in the unexpanded first configuration and then expanded from the unexpanded first configuration to the expanded second configuration in the interior of the vertebral body or in the disc space.

When inserted into the interior of the vertebral body, expansion of the spinal implant 10 presses against cancellous bone on the interior of the vertebral body, and in doing so, aids in stabilizing the vertebral body and correspondingly serves as a buttress for reinforcing cortical bone thereof. The expansion of the spinal implant 10 can also be used to push endplates of the vertebral body apart from one another to increase the height of the vertebral body. Furthermore, when inserted into the disc space between the adjacent vertebral bodies, expansion of the spinal implant 10 against endplates of the adjacent vertebral bodies can be used to restore the height of the disc space.

Whether being inserted into the interior of the vertebral body or into the disc space between the adjacent vertebral bodies, more than one of the spinal implants 10 can be used. For example, two or more of the spinal implants 10 can be inserted into the interior of the vertebral body to facilitate stabilization and reinforcement thereof, and/or increase the height of the vertebral body, and two or more of the spinal implants 10 can be inserted into the disc space between the adjacent vertebral bodies to facilitate restoration of the height of the disc space.

The spinal implant (or implants) 10 can be used with or without bone cement. For example, bone cement can be inserted into the interior of the vertebral body via the cannula and through portions of the spinal implant 10, and the spinal implant 10, when in the expanded second configuration, can serve as a lattice or framework for the bone cement before, during, and after curing thereof. Thus, use of the spinal implant 10 and the bone cement can serve to further reinforce and buttress the vertebral body from within the interior thereof.

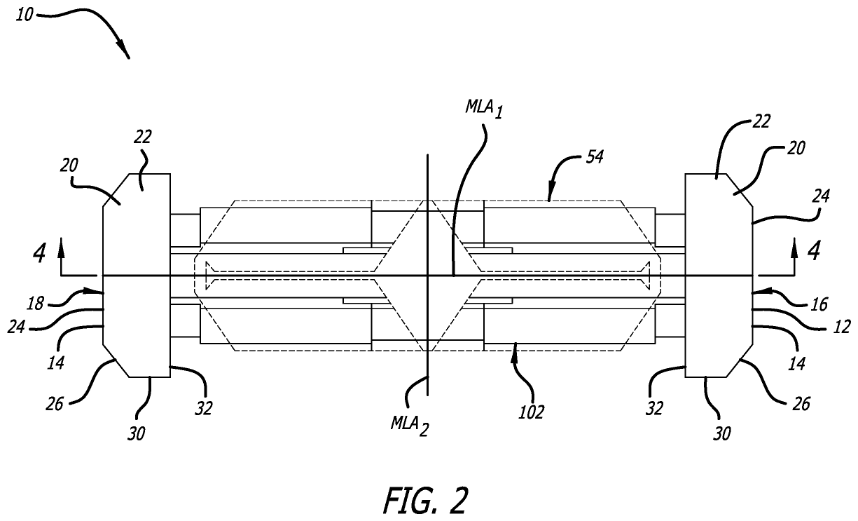

As depicted in FIGS. 1A-2, the spinal implant 10 includes a proximal first end 12, a distal second end 14, a major mid-longitudinal axis MLA₁ extending through the proximal first end 12 and the distal second end 14 in a longitudinal direction, and a minor mid-longitudinal axis MLA₂ perpendicular to the major mid-longitudinal axis MLA₁ in a lateral direction. The spinal implant 10 includes a proximal end portion 16 provided adjacent the proximal first end 12, and a distal end portion 18 provided adjacent the distal second end 14. The proximal end portion 16 and the distal end portion 18 are opposed to one another, and the proximal end portion 16 and the distal end portion 18 can be similarly or differently sized. The proximal end portion 16 and the distal end portion 18 form portions of an expander used to facilitate expansion of the spinal implant 10 from the unexpanded first configuration to the expanded second configuration.

As depicted in FIG. 1A, the proximal end portion 16 and the distal end portion 18 have generally-similar first portions 20 and second portions 22. The first portions 20 of the proximal end portion 16 and the distal end portion 18 can include end-facing surfaces 24 and beveled surfaces 26, and the second portions 22 of the proximal end portion 16 and the distal end portion 18 can include outer surfaces 30 and interior-facing surfaces 32.

In an example, the end-facing surface 24 of the proximal end portion 16 or the distal end portion 18 has a flattened or substantially-flattened shape, and the beveled surface 26 of the proximal end portion 16 or distal end portion 18 has a frusto-conical or substantially-frusto-conical shape to, for example facilitate insertion through the cannula and implantation into the interior of the vertebral body. In other examples, the end-facing surface 24 of the proximal end portion 16 or the distal end portion 18 has a convex, substantially-convex, pyramidal, or substantially-pyramidal shape. The beveled surface 26 of the proximal end portion 16 or the distal end portion 18, in such examples, have shapes that transition (e.g., change or switch shape or curvature) between the end-facing surfaces 24 and the outer surfaces 30.

The outer surface 30, for example, of the proximal end portion 16 or the distal end portion 18 can have triangular, substantially-triangular, square, substantially-square, pentagonal, substantially-pentagonal, hexagonal, substantially-hexagonal, octagonal, substantially-octagonal, etc. shape a plane perpendicular to the major mid-longitudinal axis MLA₁. Additionally, the interior-facing surface 32 can have a triangular, substantially-triangular, square, substantially-square, pentagonal, substantially-pentagonal, hexagonal, substantially-hexagonal, etc. shape. As depicted in FIGS. 1A, 5, and 6, the outer surface 30 has a substantially-octagonal shape in a plane perpendicular to the major mid-longitudinal axis MLA₁.

In some examples, the outer surface 30 of proximal end portion 16 or distal end portion 18 has a cylindrical or substantially-cylindrical shape to, for example, facilitate insertion through the cannula and implantation into the interior of the vertebral body. The interior-facing surface 32 of proximal end portion 16 or distal end portion 18, in such examples, has a corresponding circular or substantially-circular shape.

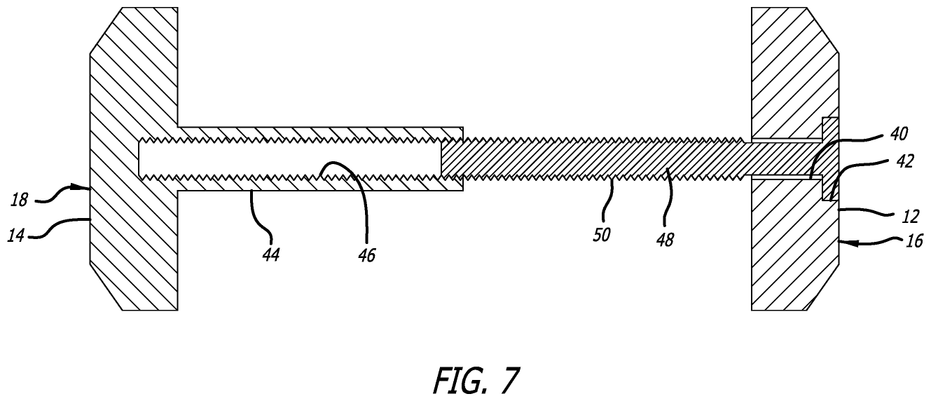

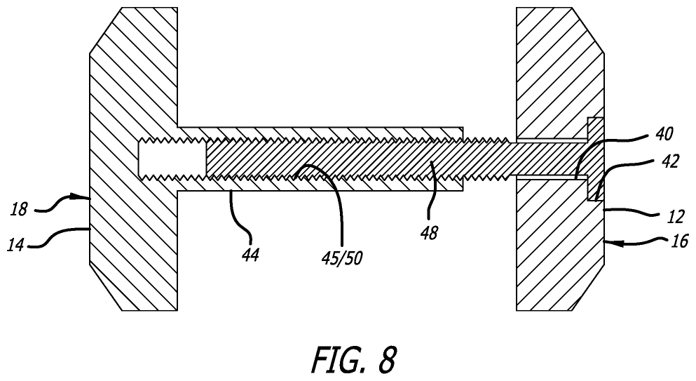

As depicted in FIGS. 7 and 8, the proximal end portion 16 includes an aperture 40 extending between the end-facing surface 24 and the interior-facing surface 32 thereof, and the aperture 40 includes a recess 42 surrounding the aperture 40 adjacent the interior-facing surface 32. Furthermore, as depicted in FIGS. 7 and 8, the distal end portion 18 includes a post 44 extending outwardly from the interior-facing surface 32, and the post 44 includes a threaded aperture 46 extending at least a portion therethrough. A screw 48 having threads 50 can be received through the aperture 40 and into the threaded aperture 46 to join to the proximal end portion 16 and the distal end portion 18. And rotation of the screw 48 and corresponding interaction between the threads 50 and the threaded aperture 46 serves to move the proximal end portion 16 and the distal end portion 18 from a first position (FIG. 7) to a second position (FIG. 8). As discussed below, movement of the proximal end portion 16 and the distal end portion 18 from the first position to the second position serves in moving the spinal implant 10 from the unexpanded first configuration (FIGS. 1A-6) to the expanded second configuration (FIGS. 9-11).

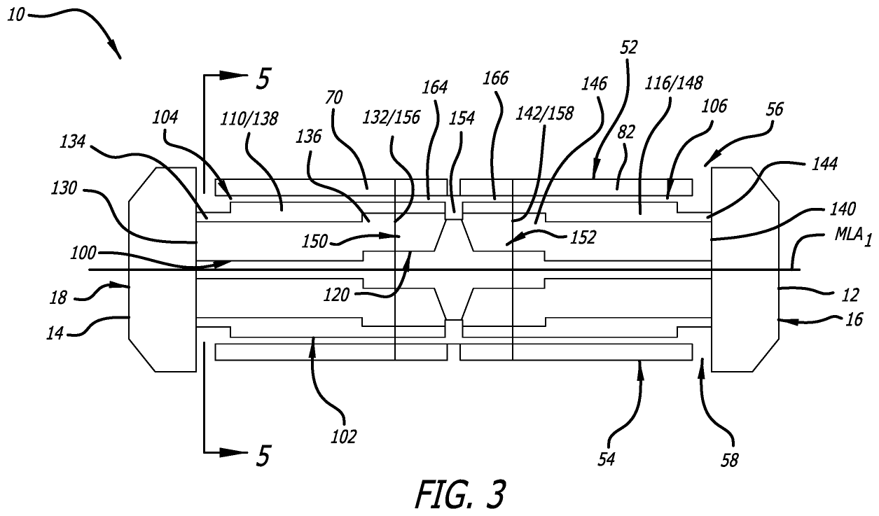

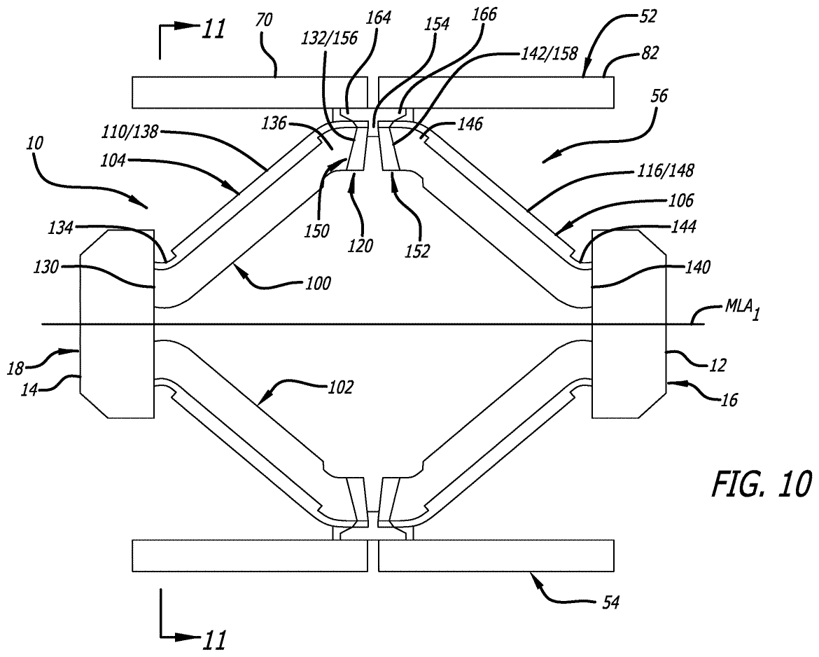

The spinal implant 10 includes a first upper expansion portion 52 (FIG. 1A) and a second lower expansion portion 54 (FIG. 2). As depicted in FIG. 3, the first upper expansion portion 52 is positioned on an upper side 56 above the major mid-longitudinal axis MLA₁, and the second lower expansion portion 54 is positioned on a lower side 58 below the major mid-longitudinal axis MLA₁. Portions of the first upper expansion portion 52 are outwardly expandable laterally (FIG. 9) and upwardly (FIG. 10) and portions of the second lower expansion portion 54 are outwardly expandable laterally (FIG. 9) and downwardly (FIG. 10) in similar fashion. As discussed further below, with the spinal implant 10 positioned in the vertebral body, the outward lateral and outward upward/downward expansion of the first upper expansion portion 52 and the second lower expansion portion 54 presses against the cancellous bone on the interior of the vertebral body to facilitate stabilization of the vertebral body. The first upper expansion portion 52 and the second lower expansion portion 54 can be mirror images of one another relative to the major mid-longitudinal axis MLA₁. As such, the features of the first upper expansion portion 52 are described in detail below, and it is to be understood that the second lower expansion portion 54 can include identical features in mirror arrangement to the features of the first upper expansion portion 52. Furthermore, portions of the first upper expansion portion 52 and the second lower expansion portion 54 (and other portions of the spinal implant 10) can include surface finishes at bone-contacting portions thereof that can be smooth or porous. Such porosity can be provided by selecting porous materials therefor or providing porous coating thereon to improve osseointegration. For example, the bone-contacting portions of the first upper expansion portion 52 and the second lower expansion portion 54 can be plasma-sprayed with a porous coating that can be, for example, metallic or made of calcium phosphate to facilitate such osseointegration,

As depicted in FIG. 1B, the first upper expansion portion 52 can include a first platform portion 60 and a second platform portion 62 positioned on a left side 64 and a right side 66, respectively, of the minor mid-longitudinal axis MLA₂. And the first platform portion 60 and the second platform portion 62 can be mirror images of one another relative to the minor mid-longitudinal axis MLA₂, and portions thereof can be moveable to facilitate lateral expansion of the first upper expansion portion 52. The first platform portion 60 can include a first arm portion 70, a second arm portion 72, and a first joint portion 74 interconnecting and pivotally attaching the first arm portion 70 and the second arm portion 72 to one another, and each of the first arm portion 70 and the second arm portion 72 can include a first end 76 and an opposite second end 78; and the second platform portion 62 can include a third arm portion 80, a fourth arm portion 82, and a second joint portion 84 interconnecting and pivotally attaching the third arm portion 80 and the fourth arm portion 82 to one another, and each of the third arm portion 80 and the fourth arm portion 82 can include a first end 86 and an opposite second end 88. The first arm portion 70, the second arm portion 72, the first joint portion 74, the third arm portion 80, the fourth arm portion 82, and/or the second joint portion 84 can be formed unitarily with one another, or formed separately and then integrated with one another.

When the proximal end portion 16 and the distal end portion 18 are moved from first position (FIG. 7) to the second position (FIG. 8), the first platform portion 60 and the second platform portion 62 would contract in the longitudinal direction and correspondingly expand outwardly in the lateral directions. During such outward lateral expansion, the first arm portion 70 and the second arm portion 72 move from an aligned position (FIG. 1B) to a V-shaped position (FIG. 9) relative to one another, and simultaneously, the third arm portion 80 and the fourth arm portion 82 move from an aligned position to a V-shaped position relative to one another. In doing so, the second end 78 of the first arm portion 70 and the second end 88 of the fourth arm portion 82 are moved outwardly laterally in a first direction relative to the major mid-longitudinal axis MLA₁ via deformation of portions of the first platform portion 60 adjacent the first joint portion 74, and the second end 78 of the second arm portion 72 and the second end 88 of the third arm portion 80 are moved outwardly laterally in an opposite second direction relative to the major mid-longitudinal axis MLA₁ via deformation of portions of the second platform portion 62 adjacent the second joint portion 84. Thus, the first joint portion 74 (and deformation at the first ends 76 of the first arm portion 70 and the second arm portion 72) affords the first arm portion 70 and the second arm portion 72 to pivot between the aligned position to the V-shaped position, and the second joint portion 84 (and deformation at the first ends 86 of the third arm portion 80 and the fourth arm portion 82) affords the third arm portion 80 and the fourth arm portion 82 to pivot between the aligned position to the V-shaped position. In doing so, the expanded area formed between the first arm portion 70 and the second arm portion 72 is expanded in the V-shaped position thereof, and the expanded area formed between the third arm portion 80 and the fourth arm portion 82 is expanded in the V-shaped position, and such expanded areas create spaces that can ultimately allow (if desired) placement of the bone cement through these expanded areas of the first upper expansion portion 52.

To facilitate outward movement of the first platform portion 60 and the second platform portion 62 (of the first upper expansion portion) and afford expansion thereof laterally and upwardly (FIGS. 9 and 10), the first upper expansion portion 52 can include an upper expandable lattice portion 100. Portions of the upper expandable lattice portion 100 can be used to expand the first platform portion 60 and the second platform portion 62 in response to movement of the proximal end portion 16 and the distal end portion 18 toward one another. In similar fashion, the second lower expansion portion 54 can include a lower expandable lattice portion 102 that can be used to expand the second lower expansion portion 54 also in response to the movement of the proximal end portion 16 and the distal end portion 18 toward one another.

The upper expandable lattice portion 100, as depicted in FIG. 1C, can include a first lattice portion 104 and a second lattice portion 106 positioned on the left side 64 and the right side 66, respectively, of the minor mid-longitudinal axis MLA₂. The first lattice portion 104 and the second lattice portion 106 can be mirror images of one another relative to the minor mid-longitudinal axis MLA₂. Furthermore, movement of the first lattice portion 104 and the second lattice portion 106 in response to movement of the proximal end portion 16 and the distal end portion 18 serves to move the platform portion 60 and the second platform portion 62 to contract in the longitudinal direction, expand in the lateral direction, and move upwardly relative to the major mid-longitudinal axis MLA₁ and the minor mid-longitudinal axis MLA_{2.}

The first lattice portion 104, as depicted in FIG. 1C, includes a first leg portion 110 and a second leg portion 112, and the second lattice portion 106 includes a third leg portion 114 and a fourth leg portion 116. Furthermore, the first leg portion 110 and the fourth leg portion 116 are attached relative to one another via a first joint portion 120, and the second leg portion 112 and the third leg portion 114 are attached relative to one another via a second joint portion 122. The first leg portion 110, the fourth leg portion 116, and/or the first joint portion 120 can be formed unitarily with one another, or formed separately and then integrated with one another. The second leg portion 112 and the third leg portion 114, and/or the second joint portion 122 can be formed unitarily with one another, or formed separately and then integrated with one another. Given that the first lattice portion 104 and the second lattice portion 106 are mirror images of one another relative to the minor mid-longitudinal axis MLA₂, the first leg portion 110 and the third leg portion 114 are similar to one another, and the second leg portion 112 and the fourth leg portion 116 are similar to one another. Furthermore, the first joint portion 120 and the second joint portion 122 are similar to one another, and are mirror images of one another relative to the major mid-longitudinal axis MLA₁.

The first leg portion 110 and the third leg portion 114 each include a first end 130, an opposite second end 132, a first end portion 134 adjacent the first end 130, an opposite second end portion 136 adjacent the second end 132, and an intermediate portion 138 extending between the first end portion 134 and the second end portion 136. The first end 130 of the first leg portion 110 is attached to the distal end portion 18, the second end 132 of the first leg portion 110 is attached to the first joint portion 120, the first end 130 of the third leg portion 114 is attached to the proximal end portion 16, and the second end 132 of the third leg portion 114 is attached to the second joint portion 122. Portions of the first end portions 134, the second end portions 136, and the intermediate portions 138 of the first leg portion 110 and the third leg portion 114 are configured to deform when the proximal end portion 16 and the distal end portion 18 are moved toward one another. The first end portions 134, the second end portions 136, and the intermediate portions 138 can have slabbed shapes, with the intermediate portions 138 having elongated shapes, and the first end portions 134 and second end portions 136 having shortened shapes that have thinner thicknesses than the intermediate portions 138. The thinner thicknesses of the first end portions 134 and the second end portions 136 relative to the intermediate portions 138 cause the first end portions 134 and the second end portions 136 to desirably deform under compression (when the proximal end portion 16 and the distal end portion 18 are moved toward one another) prior to the intermediate portions 138. The first end portions 134 and the second end portions 136 of the first leg portion 110 and the third leg portion 114, in other words, can bend and stretch/shorten under compression, and such bending and stretching/shortening thereof causes corresponding movement first end portion 110 and the third leg portion 114 relative to the major mid-longitudinal axis MLA₁ and the minor mid-longitudinal axis MLA₂. As depicted in FIG. 1C, the first leg portion 110 is attached to the distal end portion 18 and the first joint portion 120 at a canted orientation relative to the view plane of FIG. 1C, and the third leg portion 114 is attached to the proximal end portion 16 and the second joint portion 122 at a canted orientation relative to the view plane of FIG. 1C, such that, when compressed, the first end portions 134 and the second end portions 136 deform and bend to move the second end portions 136 outwardly laterally (FIG. 9) and outwardly upwardly (FIG. 10).

The second leg portion 112 and the fourth leg portion 116 each include a first end 140, an opposite second end 142, a first end portion 144 adjacent the first end 140, an opposite second end portion 146 adjacent the second end 142, and an intermediate portion 148 extending between the first end portion 144 and the second end portion 146. The first end 140 of the second leg portion 112 is attached to the distal end portion 18, the second end 142 of the second leg portion 112 is attached to the second joint portion 122, the first end 140 of the fourth leg portion 116 is attached to the proximal end portion 16, and the second end 142 of the fourth leg portion 116 is attached to the first joint portion 120. Portions of the first end portions 144, the second end portions 146, and the intermediate portions 148 of the second leg portion 112 and the fourth leg portion 116 are configured to deform when the proximal end portion 16 and the distal end portion 18 are moved toward one another. The first end portions 144, the second end portions 146, and the intermediate portions 148 can have slabbed shapes, with the intermediate portions 148 having elongated shapes, and the first end portions 144 and second end portions 146 having shortened shapes that have thinner thicknesses than the intermediate portions 148. The thinner thicknesses of the first end portions 144 and the second end portions 146 relative to the intermediate portions 148 cause the first end portions 144 and the second end portions 146 to desirably deform under compression (when the proximal end portion 16 and the distal end portion 18 are moved toward one another) prior to the intermediate portions 148. The first end portions 144 and the second end portions 146 of the second leg portion 112 and the fourth leg portion 116, in other words, can bend and stretch/shorten under compression, and such bending and stretching/shortening thereof causes corresponding movement of the second leg portion 112 and the fourth leg portion 116 relative to the major mid-longitudinal axis MLA₁ and the minor mid-longitudinal axis MLA₂. As depicted in FIG. 1C, the second leg portion 112 is attached to the distal end portion 18 and the second joint portion 122 at a canted orientation relative to the view plane of FIG. 1C, and the fourth leg portion 116 is attached to the proximal end portion 16 and the first joint portion 120 at a canted orientation relative to the view plane of FIG. 1C, such that, when compressed, the first end portions 144 and the second end portions 146 deform to move the second end portions 146 outwardly laterally (FIG. 9) and outwardly upwardly (FIG. 10).

The first joint portion 120, as depicted in FIG. 1D, is positioned between the first leg portion 110 and the fourth leg portion 116, and is configured to interconnect the first leg portion 110 and the fourth leg portion 116 to serve as a fulcrum therebetween. By serving as a fulcrum, movement of the proximal end portion 16 and the distal end portion 18 compresses the first leg portion 110 and the fourth leg portion 116, and causes the second end 132 of the first leg portion 110 and the second end 142 of the fourth leg portion 116, along with the first joint portion 120, to move outwardly laterally and upwardly relative to major mid-longitudinal axis MLA₁. As discussed below, the first arm portion 70 and the fourth arm portion 82 are attached to the first joint portion 120, and such outward lateral and upward movement of the first joint portion 120 also serves to move the second ends 78 and 88 of the first arm portion 70 and the fourth arm portion 82, respectively, outwardly laterally and upwardly to facilitate lateral expansion of the first platform portion 60 and the second platform portion 62.

The first joint portion 120, as depicted in FIG. 1D, includes a first portion 150, a second portion 152, and a connection portion 154 therebetween. The first portion 150 includes a first end 156 attached to the second end 132 of the first leg portion 110, and the second portion 152 includes a first end 158 attached to the second end 142 of the fourth leg portion 116. The first portion 150 extends toward and terminates at a second end 160, and the second portion 152 extends toward and terminates at a second end 162. The first portion 150 includes a thickness between the first end 156 and the second end 160, and the second portion 152 include a thickness between the first end 158 and the second end 162. The thickness of the first portion 150 of the first joint portion 120 can approximate the thickness of the second end portion 136 of the first leg portion 110, and the thickness of the second portion 152 of the first joint portion 120 can approximate the thickness of the second end portion 146 of the fourth leg portion 116. Like the second end portion 136 (of the first leg portion 110) and the second end portion 146 (of the fourth leg portion 116), the first portion 150 and the second portion 152 can bend and stretch/shorten under compression. The connection portion 154 connects and extends between the second end 160 of the first portion 150 and the second end 162 of the second portion 152, and allows the compressive forces generated by movement of the proximal end portion 16 and the distal end portion 18 toward one another to be transferred between the first portion 150 and the second portion 152, and correspondingly between the first leg portion 110 and the fourth leg portion 116.

As such, the connection portion 154 forces the first leg portion 110, the fourth leg portion 116, the first portion 150, and the second portion 152 to be compressed when the proximal end portion 16 and the distal end portion 18 are moved toward one another. Furthermore, the first portion 150 and the second portion 152 include a first attachment portion 164 and a second attachment portion 166, respectively. The first attachment portion 164 is a strip of material that attaches the first portion 150 of the first joint portion 120 to the first arm portion 70, and the second attachment portion 166 is a strip of material that attaches the second portion 152 of the first joint portion 120 to the fourth arm portion 82. Thus, as the proximal end portion 16 and the distal end portion 18 are moved toward the second position with respect to one another, the first leg portion 110, the fourth leg portion 116, the first portion 150, and the second portion 152 are compressed that correspondingly moves the second end 132 of the first leg portion 110 outwardly laterally and upwardly, moves the second end 142 of the fourth leg portion 116 outwardly laterally and upwardly, and moves the first joint portion 120 outwardly laterally and upwardly. Given the connection afforded by the first attachment portion 164 to the first arm portion 70, the outward lateral and upward movement of the second end 132 moves the second end 78 of the first arm portion 70 outwardly laterally and upwardly to correspondingly expand a portion of the first platform portion 60 (FIG. 9); and given the connection afforded by the second attachment portion 166 to the fourth arm portion 82, the outward lateral and upward movement of the second end 142 moves the second end 88 of the fourth arm portion 82 outwardly laterally and upwardly to correspondingly expand a portion of the second platform portion 62 (FIG. 9).

The second joint portion 122, as depicted in FIG. 1D, is positioned between the second leg portion 112 and the third leg portion 114, and is configured to interconnect the second leg portion 112 and the third leg portion 114 to serve as a fulcrum therebetween. By serving as a fulcrum, movement of the proximal end portion 16 and the distal end portion 18 compresses the second leg portion 112 and the third leg portion 114, and causes the second end 142 of the second leg portion 112 and the second end 132 of the third leg portion 114, along with the second joint portion 122, to move outwardly laterally and upwardly relative to major mid-longitudinal axis MLA₁. As discussed below, the second arm portion 72 and the third arm portion 80 are attached to the second joint portion 122, and such outward lateral and upward movement of the second joint portion 122 also serves to move the second ends 78 and 88 of the second arm portion 72 and the third arm portion 80, respectively, outwardly laterally and upwardly to facilitate lateral expansion of the first platform portion 60 and the second platform portion 62.

The second joint portion 122, as depicted in FIG. 1E, includes a first portion 170, a second portion 172, and a connection portion 174 therebetween. The first portion 170 includes a first end 176 attached to the second end 142 of the second leg portion 112, and the second portion 172 includes a first end 178 attached to the second end 132 of the third leg portion 114. The first portion 170 extends toward and terminates at a second end 180, and the second portion 172 extends toward and terminates at a second end 182. The first portion 170 includes a thickness between the first end 176 and the second end 180, and the second portion 172 include a thickness between the first end 178 and the second end 182. The thickness of the first portion 170 of the second joint portion 122 can approximate the thickness of the second end portion 146 of the second leg portion 112, and the thickness of the second portion 172 of the second joint portion 122 can approximate the thickness of the second end portion 136 of the third leg portion 114. Like the second end portion 146 (of the second leg portion 112) and the second end portion 136 (of the third leg portion 114), the first portion 170 and the second portion 172 can bend and stretch/shorten under compression. The connection portion 174 connects and extends between the second end 180 of the first portion 170 and the second end 182 of the second portion 172, and allows the compressive forces generated by movement of the proximal end portion 16 and the distal end portion 18 toward one another to be transferred between the first portion 170 and the second portion 172, and correspondingly between the second leg portion 112 and the third leg portion 114.

As such, the connection portion 174 forces the second leg portion 112, the third leg portion 114, the first portion 170, and the second portion 172 to be compressed when the proximal end portion 16 and the distal end portion 18 are moved toward one another. Furthermore, the first portion 170 and the second portion 172 include a first attachment portion 184 and a second attachment portion 186, respectively. The first attachment portion 184 is a strip of material that attaches the first portion 170 of the second joint portion 122 to the second arm portion 72, and the second attachment portion 186 is a strip of material that attaches the second portion 172 of the second joint portion 122 to the third arm portion 80. Thus, as the proximal end portion 16 and the distal end portion 18 are moved toward the second position with respect to one another, the second leg portion 112, the third leg portion 114, the first portion 170, and the second portion 172 are compressed that correspondingly moves the second end 142 of the second leg portion 112 outwardly laterally and upwardly, moves the second end 132 of the third leg portion 114 outwardly laterally and upwardly, and moves the second joint portion 122 outwardly laterally and upwardly. Given the connection afforded by the first attachment portion 184 to the second arm portion 72, the outward lateral and upward movement of the second end 142 moves the second end 78 of the second arm portion 72 outwardly laterally and upwardly to correspondingly expand a portion of the first platform portion 60 (FIG. 9); and given the connection afforded by the second attachment portion 186 to the third arm portion 80, the outward lateral and upward movement of the second end 132 moves the second end 88 of the third arm portion 80 outwardly laterally and upwardly to correspondingly expand a portion of the second platform portion 62 (FIG. 9).

The use of the upper expandable lattice portion 100, the first joint portion 74, the second joint portion 84, the connections of the first arm portion 70 and the fourth arm portion 82 to the first joint portion 120 (afforded by the first attachment portion 164 and the second attachment portion 166), and the connections of the second arm portion 72 and the third arm portion 74 to the second joint portion 122 (afforded by the first attachment portion 184 and the second attachment portion 186), affords pivoting of the first arm portion 70 and the second arm portion 72 from the aligned position to the V-shaped position and pivoting of the third arm portion 80 and the fourth arm portion 82 from the aligned position to the V-shaped position as the proximal end portion 16 and the distal end portion 18 are moved from the first position (FIG. 7) toward the second position (FIG. 8) with respect to one another. Thus, when the proximal end portion 16 and the distal end portion 18 are moved toward one another to move the spinal implant 10 from the unexpanded first configuration to the expanded second configuration, the first joint portion 74 affords the first arm portion 70 and the second arm portion 72 to pivot between the aligned position to the V-shaped position, and the second joint portion 84 affords the third arm portion 80 and the fourth arm portion 82 to pivot between the aligned position to the V-shaped position. In doing so, the area formed between the first arm portion 70 and the second arm portion 72 is expanded in the V-shaped position thereof, and the area formed between the third arm portion 80 and the fourth arm portion 82 is expanded in the V-shaped position, and such expanded areas create spaces that ultimately allow placement of the bone cement through these expanded areas of the first upper expansion portion 52.

After insertion of the spinal implant 10 into the vertebral body, the first upper expansion portion 52 and the mirror-image analog thereof, the second lower expansion portion 54, can be expanded within the vertebral body to serve in stabilizing the vertebral body from the inside. Then, the bone cement (if desired) can be inserted into the interior of the vertebral body and through portions of the spinal implant 10. In doing so, the spinal implant 10, when in the expanded second configuration, can serve as a lattice or framework for the bone cement before, during, and after curing thereof.

The componentry of the spinal implant 10 including the componentry of the first upper expansion portion 52 and the second lower expansion portion 54 can be made of metallic and/or non-metallic materials. The materials used for the componentry of the spinal implant 10 can afford the above-discussed deformation, for example, of the first end portions 134 and the second end portions 136 of the first leg portion 110 and the third leg portion 114, the first end portions 144 and the second end portions 146 of the second leg portion 112 and the fourth leg portion 116, the first ends 76 of the first arm portion 70 and the second arm portion 72, the first ends 86 of the third arm portion 80 and the fourth arm portion 82, the first portion 150 and the second portion 152 of the first joint portion 120, and the first portion 170 and the second portion 172 of the second joint portion 122. The materials can be selected so that similar componentry deform at similar rates during compression thereof caused by the movement of the proximal end portion 16 and the distal end portion 18 toward one another. Furthermore, the deformation can be elastic or plastic, and the materials can be selected to facilitate such elastic or plastic deformation.

It should be understood that various aspects disclosed herein may be combined in different combinations than the combinations specifically presented in the description and the accompanying drawings. It should also be understood that, depending on the example, certain acts or events of any of the processes of methods described herein may be performed in a different sequence, may be added, merged, or left out altogether (e.g., all described acts or events may not be necessary to carry out the techniques). In addition, while certain aspect of this disclosure are described as being performed by a single module or unit for purposes of clarity, it should be understood that the techniques of this disclosure may be performed by a combination of units or modules associated with, for example, a medical device.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The following examples relate to the present disclosure
1. An expandable spinal implant comprising:
   a distal first end, an opposite proximal second end, a major mid-longitudinal axis extending through the distal first end and the proximal second end, and a minor mid-longitudinal axis extending perpendicularly to the mid-longitudinal axis, a first reference plane extending through and along the major mid-longitudinal axis;
   a distal end portion adjacent the distal first end, and a proximal end portion adjacent the proximal first end, the distal end position and the proximal portion being moveable toward one another;
   a first leg portion, a second leg portion, a third leg portion, and a fourth leg portion, each of the first leg portion, the second leg portion, the third leg portion, and the fourth leg portion including a first end and an opposite second end,
   the first end of the first leg portion being attached relative to the distal end portion on a first side of the first plane,
   the first end of the second leg portion being attached relative to the distal end portion on a second side of the first plane,
   the first end of the third leg portion being attached relative to the proximal end portion on the second side of the first plane, and
   the first end of the fourth leg portion being attached relative to the proximal end portion on the first side of the first plane,
   a first joint portion positioned between the first leg portion and the fourth leg portion, the second ends of the first leg portion and the fourth leg portion being attached on opposite sides of the first joint portion,
   a second joint portion positioned between the second leg portion and the third leg portion, the second ends of the second leg portion and the third leg portion being attached on opposite sides of the second joint portion,
   a first arm portion, a second arm portion, a third arm portion, and a fourth arm portion, each of the first arm portion, the second arm portion, the third arm portion, and the fourth arm portion including a first end and an opposite second end,
   the first ends of the first arm portion and the second arm portion being attached relative to one another,
   the first ends of the third arm portion and the fourth arm portion being attached relative to one another,
   the second ends of the first arm portion and the fourth arm portion being attached relative to the first joint portion, and
   the second ends of the second arm portion and the third arm portion being attached relative to the second joint portion,
   wherein, when the distal end portion and the proximal end portion are moved toward one another,
   the first joint portion is displaced outwardly in a first lateral direction and upwardly relative to the major mid-longitudinal axis,
   the second joint portion is displaced outwardly in a second lateral direction and upwardly relative to the major mid-longitudinal axis; and
   wherein outward and upward movement of the first joint portion moves the second ends of the first arm portion and the fourth arm portion outwardly in the first lateral direction and upwardly, and outward and upward movement of the second joint portion moves the second ends of the second arm portion and the third arm portion outwardly in the second lateral direction and upwardly,
   the outward and upward movement of the second end of the first arm portion and the outward and upward movement of the second end of the second arm portion moving the second ends of the first arm portion and the second arm portion apart from one another, and
   the outward and upward movement of the second end of the third arm portion and the outward and upward movement of the second end of the fourth arm portion moving the second ends of the third arm portion and the fourth arm portion apart from one another.
2. The expandable spinal implant of example 1, wherein the first leg portion, the second leg portion, the third leg portion, the fourth leg portion, the first joint portion, the second joint portion, the first arm portion, the second arm portion, the third arm portion, and the fourth arm portion form an upper portion of the expandable spinal implant, and further comprising a lower portion of the expandable spinal implant including a fifth leg portion, a sixth leg portion, a seventh leg portion, an eighth leg portion, a third joint portion, a fourth joint portion, a fifth arm portion, a sixth arm portion, a seventh arm portion, and an eighth arm portion arranged similarly to the first leg portion, the second leg portion, the third leg portion, the fourth leg portion, the first joint portion, the second joint portion, the first arm portion, the second arm portion, the third arm portion, and the fourth arm portion form an upper portion of the expandable spinal implant, respectively.
3. The expandable spinal implant of example 1, wherein the outward and upward movement of the second end of the first arm portion, the outward and upward movement of the second end of the second arm portion, the outward and upward movement of the second end of the third arm portion, and the outward and upward movement of the second end of the fourth arm portion moves the expandable spinal implant from an unexpanded first configuration to an expanded second configuration.
4. The expandable spinal implant of example 3, wherein, when the expandable spinal implant is in the unexpanded first configuration, the first arm portion and the second arm portion are in a aligned position with respect to one another, and when the expandable spinal implant is in the expanded second configuration, the first arm portion and the second arm portion are in a V-shaped position with respect to one another.
5. The expandable spinal implant of example 4, wherein, when the expandable spinal implant is in the unexpanded first configuration, the third arm portion and the fourth arm portion are in an aligned position with respect to one another, and when the expandable spinal implant is in the expanded second configuration, the third arm portion and the fourth arm portion are in a V-shaped position with respect to one another.
6. The expandable spinal implant of example 5, wherein the first ends of the first arm portion and the second arm portion are pivotally attached relative to one another, and the first ends of the third arm portion and the fourth arm portion are pivotally attached relative to one another.
7. The expandable spinal implant of example 5, wherein after implantation into a vertebral body, and when the first arm portion and the second arm portion are in the V-shaped position, and the third arm portion and the fourth arm portion are in the V-shaped position, an area is formed between the first arm portion, the second arm portion, third arm portion, and the fourth arm portion form an aperture affording bone growth therethrough.
8. The expandable spinal implant of example 1, wherein during movement of the distal end portion and the proximal end portion toward one another, the first leg portion and the fourth leg portion are compressed thereby forcing the second ends thereof outwardly and upwardly.
9. The expandable spinal implant of example 8, wherein during movement of the distal end portion and the proximal end portion toward one another, the second leg portion and the third leg portion are compressed thereby forcing the second ends thereof outwardly and upwardly.
10. The expandable spinal implant of example 1, wherein one of the distal end portion and the proximal end portion includes an aperture therethrough, and the other of the distal end portion and the proximal end portion includes a post portion with a threaded aperture therein, and further comprising a threaded screw receivable through the aperture and into the threaded aperture in the post portion to attach the distal end portion and the proximal end portion to one another.
11. The expandable spinal implant of example 10, wherein, when the threaded screw is received through the aperture and into the threaded aperture, rotation of the threaded screw in a first direction moves the distal end portion and the proximal end portion toward one another.
12. An expandable spinal implant configured to move from an unexpanded first configuration to an expanded second configuration, the expandable spinal implant comprising:
   a distal first end, an opposite proximal second end, a major mid-longitudinal axis extending through the distal first end and the proximal second end, and a minor mid-longitudinal axis extending perpendicularly to the mid-longitudinal axis;
   a distal end portion adjacent the distal first end, and a proximal end portion adjacent the proximal first end, the distal end position and the proximal portion being moveable toward one another;
   a first leg portion, a second leg portion, a third leg portion, and a fourth leg portion each including a first end and an opposite second end,
   the first end of the first leg portion being attached relative to a first portion of the distal end portion,
   the first end of the second leg portion being attached relative to a second portion of the distal end portion,
   the first end of the third leg portion being attached relative to a first portion of the proximal end portion, and
   the first end of the fourth leg portion being attached relative to a second portion of the proximal end portion,
   a first joint portion positioned between the first leg portion and the fourth leg portion, the second ends of the first leg portion and the fourth leg portion being attached on opposite sides of the first joint portion,
   a second joint portion positioned between the second leg portion and the third leg portion, the second ends of the second leg portion and the third leg portion being attached on opposite sides of the second joint portion,
   a first arm portion, a second arm portion, a third arm portion, and a fourth arm portion each including a first end and an opposite second end,
   the first ends of the first arm portion and the second arm portion being pivotally attached relative to one another,
   the first ends of the third arm portion and the fourth arm portion being pivotally attached relative to one another,
   the second ends of the first arm portion and the fourth arm portion being attached relative to the first joint portion, and
   the second ends of the second arm portion and the third arm portion being attached relative to the second joint portion,
   wherein, when the distal end portion and the proximal end portion are moved toward one another,
   the first joint portion is forced outwardly in a first lateral direction and upwardly relative to the major mid-longitudinal axis due to compression of the first leg portion and the fourth leg portion between the distal end portion and the proximal end portion, and
   the second joint portion is forced outwardly in a second lateral direction and upwardly relative to the major mid-longitudinal axis due to compression of the second leg portion and the third leg portion between the distal end portion and the proximal end portion; and
   wherein outward and upward movement of the first joint portion moves the second ends of the first arm portion and the fourth arm portion outwardly in the first lateral direction and upwardly, and outward and upward movement of the second joint portion moves the second ends of the second arm portion and the third arm portion outwardly in the second lateral direction and upwardly, movement of the first ends of the first arm portion and the fourth arm portion, and movement of the first ends of the second arm portion and the third arm portion moves the expandable spinal implant from the unexpanded first configuration to the expanded second configuration.
13. The expandable spinal implant of example 12, wherein the first leg portion, the second leg portion, the third leg portion, the fourth leg portion, the first joint portion, the second joint portion, the first arm portion, the second arm portion, the third arm portion, and the fourth arm portion form an upper portion of the expandable spinal implant, and further comprising a lower portion of the expandable spinal implant including a fifth leg portion, a sixth leg portion, a seventh leg portion, an eighth leg portion, a third joint portion, a fourth joint portion, a fifth arm portion, a sixth arm portion, a seventh arm portion, and an eighth arm portion arranged similarly to the first leg portion, the second leg portion, the third leg portion, the fourth leg portion, the first joint portion, the second joint portion, the first arm portion, the second arm portion, the third arm portion, and the fourth arm portion form an upper portion of the expandable spinal implant, respectively.
14. The expandable spinal implant of example 12, wherein, when the expandable spinal implant is in the unexpanded first configuration, the first arm portion and the second arm portion are in a aligned position with respect to one another, and when the expandable spinal implant is in the expanded second configuration, the first arm portion and the second arm portion are in a V-shaped position with respect to one another.
15. The expandable spinal implant of example 14, wherein, when the expandable spinal implant is in the unexpanded first configuration, the third arm portion and the fourth arm portion are in an aligned position with respect to one another, and when the expandable spinal implant is in the expanded second configuration, the third arm portion and the fourth arm portion are in a V-shaped position with respect to one another.
16. The expandable spinal implant of example 15, wherein after implantation into a vertebral body, and when the first arm portion and the second arm portion are in the V-shaped position, and the third arm portion and the fourth arm portion are in the V-shaped position, an area is formed between the first arm portion, the second arm portion, third arm portion, and the fourth arm portion form an aperture affording bone growth therethrough.
17. The expandable spinal implant of example 12, wherein one of the distal end portion and the proximal end portion includes an aperture therethrough, and the other of the distal end portion and the proximal end portion includes a post portion with a threaded aperture therein, and further comprising a threaded screw receivable through the aperture and into the threaded aperture in the post portion to attach the distal end portion and the proximal end portion to one another.
18. The expandable spinal implant of example 17, wherein, when the threaded screw is received through the aperture and into the threaded aperture, rotation of the threaded screw in a first direction moves the distal end portion and the proximal end portion toward one another.
19. An expandable spinal implant configured to be move from an unexpanded first configuration to an expanded second configuration, the expandable spinal implant comprising:
   a distal first end, an opposite proximal second end, a major mid-longitudinal axis extending through the distal first end and the proximal second end, and a minor mid-longitudinal axis extending perpendicularly to the mid-longitudinal axis;
   a distal end portion adjacent the distal first end, and a proximal end portion adjacent the proximal first end, the distal end position and the proximal portion being moveable toward one another;
   a first leg portion, a second leg portion, a third leg portion, and a fourth leg portion each including a first end and an opposite second end,
   the first end of the first leg portion being attached relative to the distal end portion,
   the first end of the second leg portion being attached relative to the distal end portion,
   the first end of the third leg portion being attached relative to the proximal end portion, and
   the first end of the fourth leg portion being attached relative to the proximal end portion,
   a first joint portion including a first side portion and a second side portion, the second ends of the first leg portion and the fourth leg portion being respectively attached relative to the first side portion and the second side portion of the first joint portion,
   a second joint portion including a first side portion and a second side portion, the second ends of the second leg portion and the third leg portion being respectively attached relative to the first side portion and the second side portion of the second joint portion,
   a first arm portion, a second arm portion, a third arm portion, and a fourth arm portion each including a first end and an opposite second end,
   the first ends of the first arm portion and the second arm portion being attached relative to one another,
   the first ends of the third arm portion and the fourth arm portion being attached relative to one another,
   the second ends of the first arm portion and the fourth arm portion being attached relative to the first joint portion, and
   the second ends of the second arm portion and the third arm portion being attached relative to the second joint portion,
   wherein, when the distal end portion and the proximal end portion are moved toward one another,
   the first joint portion is forced outwardly in a first lateral direction and upwardly relative to the major mid-longitudinal axis, and
   the second joint portion is forced outwardly in a second lateral direction and upwardly relative to the major mid-longitudinal axis; and
   wherein outward and upward movement of the first joint portion moves the second ends of the first arm portion and the fourth arm portion outwardly in the first lateral direction and upwardly, and outward and upward movement of the second joint portion moves the second ends of the second arm portion and the third arm portion outwardly in the second lateral direction and upwardly, movement of the first ends of the first arm portion and the fourth arm portion, and movement of the first ends of the second arm portion and the third arm portion moves the expandable spinal implant from the unexpanded first configuration to the expanded second configuration.
20. The expandable spinal implant of example 19, wherein the first leg portion, the second leg portion, the third leg portion, the fourth leg portion, the first joint portion, the second joint portion, the first arm portion, the second arm portion, the third arm portion, and the fourth arm portion form an upper portion of the expandable spinal implant, and further comprising a lower portion of the expandable spinal implant including a fifth leg portion, a sixth leg portion, a seventh leg portion, an eighth leg portion, a third joint portion, a fourth joint portion, a fifth arm portion, a sixth arm portion, a seventh arm portion, and an eighth arm portion arranged similarly to the first leg portion, the second leg portion, the third leg portion, the fourth leg portion, the first joint portion, the second joint portion, the first arm portion, the second arm portion, the third arm portion, and the fourth arm portion form an upper portion of the expandable spinal implant, respectively.

1-20. (canceled)

21. A device for treatment of a vertebral body, the device comprising:
an implantable device comprising:
a proximal member and a distal member operatively coupled to one another,
first and second supporting structures extending between the proximal member and the distal member, each of the first and second supporting structures having respective first end portions and second end portions, each of the first end portions of the first and second supporting structures connected to one of the proximal member or the distal member, and
first and second platform structures disposed above the first supporting structure and below the second supporting structure, respectively, each of the first and second platform structures having respective end portions, wherein the end portions of the first and second platform structures are connected to the second end portions of the first and second supporting structures, respectively, and
wherein moving the proximal and distal members toward each other causes the first and second platform structures to move away from each other and the second end portions of the first and second supporting structures to move away from each other.

22. The device of claim 21, wherein moving the proximal and distal members towards each other causes the first and second platform structures to move away from each other along a first dimension and the second end portions of the first and second supporting structures to move away from each other along a second dimension, wherein the second dimension is substantially perpendicular to the first dimension.

23. The device of claim 21, wherein the implantable device further comprises a plurality of connectors, the plurality of connectors connecting the end portions of the first and second platform structures to the second end portions of the first and second supporting structures, respectively.

24. The device of claim 23, wherein the plurality of connectors are flexible.

25. The device of claim 21, wherein the first end portions of the first and second supporting structures are flexible.

26. The device of claim 21, wherein the first supporting structure comprises a plurality of first legs and the second supporting structure comprises a plurality of second legs.

27. The device of claim 26, wherein each leg of the plurality of first legs and the plurality of second legs has a first end portion and a second end portion, each first end portion connected to the proximal member or to the distal member.

28. The device of claim 26, wherein each leg of the plurality of first legs and the plurality of second legs has a first end portion and a second end portion, each second end portion connected to one of the end portions of the first and second platform structures.

29. The device of claim 21, wherein the first platform structure comprises a plurality of first arms and the second platform structure comprises a plurality of second arms.

30. The device of claim 29, wherein at least two arms of the plurality of first arms are connected via a first joint and/or at least two arms of the plurality of second arms are connected via a second joint.

31. A device for treatment of a vertebral body, the device comprising:
an implantable device comprising:
a proximal member and a distal member operatively coupled to one another, first and second platform structures, and
a supporting structure coupled to the proximal and distal members and to the first and second platform structures, and
wherein when the proximal and distal members are moved toward each other, the implantable device transforms from a collapsed configuration towards an expanded configuration such that the first and second platform structures move apart from one another and the supporting structure expands such that a cross-sectional area enclosed by the first and second platform structures increases.

32. The device of claim 31, wherein the proximal member comprises a first extension portion, and wherein the distal member comprises a second extension portion having a lumen, the lumen configured to receive the first extension portion.

33. The device of claim 32, wherein the first extension portion is a screw and the second extension portion is a post having a threaded aperture.

34. The device of claim 33, wherein the proximal and distal members are configured to move toward each other via rotation of the screw in a first direction and move away from one another via rotation of the screw in a second direction opposite the first direction.

35. The device of claim 31, wherein the device further comprises an elongate member, the elongate member having a proximal end region and a distal end region, the distal end region of the elongate member configured to be positioned within the vertebral body, wherein the implantable device is disposed at the distal end region of the elongate member.

36. The device of claim 35, wherein, in the expanded configuration, the implantable device encloses an interior volume.

37. The device of claim 36, wherein the elongate member is configured to deliver bone cement to the interior volume.

38. The device of claim 31, wherein the implantable device further comprises a plurality of connectors, the plurality of connectors configured to couple the supporting structure to the first and second platform structures.

39. The device of claim 38, wherein the plurality of connectors comprise flexible connectors.

40. A device for treatment of a vertebral body, the device comprising:
an implantable device comprising:
a proximal member and a distal member operatively coupled to one another,
first and second supporting structures extending between the proximal member and the distal member, each of the first and second supporting structures having respective first end portions and second end portions, each of the first end portions of the first and second supporting structures connected to one of the proximal member or the distal member, and
first and second platform structures disposed above the first supporting structure and below the second supporting structure, respectively, each of the first and second platform structures having respective end portions, wherein the end portions of the first and second platform structures are connected to the second end portions of the first and second supporting structures, respectively, via a plurality of connectors, and
wherein compression of the implantable device along a first dimension causes expansion of the implantable device in second and third dimensions.
